(19) United States
(12) Patent Application Publication
(10) Pub. No.: US 2026/0151607 A1
(43) Pub. Date: Jun. 4, 2026

(54) SKIN TREATMENT DEVICE

(71) Applicant: CHUNGWOO MEDICAL CO., LTD., Seoul (KR)

(72) Inventors: IL-KWON LEE, Anyang-si (KR); KEE-SEOK LEE, Seoul (KR)

(21) Appl. No.: 18/985,342

(22) Filed: Dec. 18, 2024

(30) Foreign Application Priority Data
Dec. 2, 2024 (KR) 10-2024-0176614

(51) Int. Cl. A61M 37/00

(52) U.S. Cl. CPC A61M 37/0015; A61M 2205/33

(57) ABSTRACT

The present disclosure relates to a skin treatment device including: a handpiece assembly: a needle assembly separably coupled to the handpiece assembly and having a plurality of needles exposedly passing through the end of the handpiece assembly and a pair of bases loaded with springs; a driver located inside the handpiece assembly to allow the needles to exposedly pass through the end of the handpiece assembly by desired distances according to a given signal; and a controller electrically connected to the needles of the needle assembly.

### BACKGROUND OF THE DISCLOSURE

### Cross Reference to Related Application of the Disclosure

The present application claims the benefit of Korean Patent Application No.10-2024-0176614 filed in the Korean Intellectual Property Office on Dec. 2, 2024, the entire contents of which are incorporated herein by reference.

### Field of the Disclosure

The present disclosure relates to a skin treatment device, more specifically to a skin treatment device that is capable of preventing large-area skin from being displaced at the time when needles penetrate the skin and allowing the penetration depths of the needles to be consistently controlled, thereby obtaining efficient skin treatment through the skin regeneration caused by radio frequency application.

### Background of the Related Art

To solve skin problems such as skin tissue aging, scars, acne marks, etc., generally, there is suggested a method for simply applying medicine for skin regeneration such as solution, cream, gel, ointment, and the like to the skin.

In the total amount of the medicine such as solution, cream, gel, ointment, and the like applied to the skin, however, an amount of the medicine absorbed into the dermis of the skin and thus used efficiently for the treatment of the skin is extremely small, which results in the failure in efficient skin tissue regeneration.

Therefore, there is a need to develop a method for remarkably increasing an absorption rate of the medicine such as solution, cream, gel, ointment, and the like into the skin for efficient skin tissue regeneration, and according to such a need, there are disclosed Korean Patent No. 10-0856736, Korean Patent No. 10-1181870, and Korean Patent No. 10-1363000.

Referring to the prior art patent documents, however, radio frequencies are not applied to large-area skin so that skin treatment through skin regeneration is not obtained, and further, the skin is displaced at the time when needles penetrate the skin. Besides, the penetration depths of the needles are not consistently controlled, and layers of the skin having different depths such as the papillary dermis and the reticular dermis are not simultaneously treated. Further, radio frequency application and medicine injection are not simultaneously performed, and skin pain and damage occur upon the application of electrical signals.

### Prior Art Document

### Patent Documents

1) Korean Patent No. 10-0856736 (Sep. 4, 2008)
2) Korean Patent No. 10-1181870 (Sep. 11, 2012)
3) Korean Patent No. 10-1363000 (Feb. 24, 2014)

### SUMMARY OF THE DISCLOSURE

Accordingly, the present disclosure has been made in view of the above-mentioned problems occurring in the related art, and it is an object of the present disclosure to provide a skin treatment device that is capable of allowing a handpiece assembly to have a negative pressure-forming portion formed on the end thereof in the form of a long ditch and/or manholes positioned at given intervals around needles, thereby preventing large-area skin from being displaced at the time when the needles penetrate the skin, allowing the penetration depths of the needles to be consistently controlled, preventing skin pain and damage from occurring upon radio frequency application, and obtaining efficient skin treatment through the skin regeneration caused by the radio frequency application.

It is another object of the present disclosure to provide a skin treatment device that is capable of simultaneously treating the layers of the skin having different depths such as the papillary dermis and the reticular dermis.

It is yet another object of the present disclosure to provide a skin treatment device that is capable of simultaneously performing radio frequency application and medicine injection.

To accomplish the above-mentioned objects, according to the present disclosure, there is provided a skin treatment device including: a handpiece assembly: a needle assembly separably coupled to the handpiece assembly and having a plurality of needles exposedly passing through the end of the handpiece assembly and a pair of bases loaded with springs; a driver located inside the handpiece assembly to allow the needles to exposedly pass through the end of the handpiece assembly by desired distances according to a given signal; and a controller electrically connected to the needles of the needle assembly, wherein the springs may allow the needles to exposedly pass through the end of the handpiece assembly upon the movement of the needle assembly, thereby keeping conductive states of the needles, the needles being exposed in the parallel direction to the moving direction of the needle assembly, the handpiece assembly may include negative pressure-forming portions formed on the end thereof to suck the skin, while applying negative pressure to a space made with the contact with the skin around the needles, so that while the needles are exposed and thus penetrate the skin, the skin is fixed, without being displaced, and the needle assembly may further include a silicone cover adapted to keep air tightness and thus prevent the negative pressure formed through the negative pressure-forming portions from leaking to the spaces passing through the needles.

According to the present disclosure, desirably, the handpiece assembly may include metal members located on the end thereof in such a way as to come into close contact with the skin, together with the negative pressure-forming portions, and thus cool the skin.

According to the present disclosure, desirably, some of the needles may be exposed longer than the remaining needles and thus different in skin penetration depths.

According to the present disclosure, desirably, some of the needles may be hollow so that the needles may penetrate the skin and inject medicine into the skin.

According to the present disclosure, desirably, if some of the needles may be hollow so that the needles may inject the medicine into the skin, the medicine may be fed from a storage tank of a medicine injection part that is detachably mounted on the handpiece assembly, pushed through a pushing rod located in the storage tank, and injected into the skin, the pushing rod being coupled to a motor of the medicine injection part in such a way as to operate through the controller connected thereto so that the medicine may be pushed and injected at consistent time and speed.

According to the present disclosure, desirably, the handpiece assembly may be provided with a vibrator that applies vibrations so that the injected medicine may uniformly spread.

According to the present disclosure, desirably, the vibrations may include vibrations in the same directions as the exposed directions of the needles and vibrations in diagonal directions with respect to the exposed directions of the needles.

According to the present disclosure, desirably, the entire portion excepting the ends of some or all of the needles may be insulatedly formed.

According to the present disclosure, desirably, in the application of electrical signals to the needles from the controller, the controller may adopt a bipolar method in which positive poles are connected to some of the needles and negative poles are connected to the remaining needles.

According to the present disclosure, desirably, in the application of electrical signals to the needles from the controller, the controller may adopt a monopolar method in which positive poles are connected to the plurality of needles and negative poles are connected separately to a given area of the human body.

According to the present disclosure, desirably, the handpiece assembly may further include a locker adapted to restrict the needle assembly to allow the needle assembly to be coupled thereto and released from the coupled state.

According to the present disclosure, desirably, the needle assembly may include a storage unit through which unique identifiable information is obtained, and the information stored in the storage unit may include a manufacturing date, the number of pins, the number of uses, an expiration date, and version information.

According to the present disclosure, desirably, the needle assembly may have the unique identifiable information in the form of a barcode or QR code.

According to the present disclosure, desirably, the handpiece assembly or the needle assembly may have an LED built therein so that lighting may be controlled by the signal provided from the controller electrically connected to the handpiece assembly or the needle assembly to allow the light emitted from the LED to be applied to the area to be treated through the end of the handpiece assembly, and the colors of the LED may represent the operating states of the skin treatment device so that the application of the electrical signals to the human body through the needles may be displayed.

According to the present disclosure, desirably, the needles may be exposed by the given distances and thus apply the electrical signals to the skin, and the needles may have different penetration depths on the same positions and thus reach one or more depths to apply the electrical signals.

According to the present disclosure, desirably, the needles may be electrically coupled to one another and thus apply the electrical signals from the controller for a desired time to the skin, and the electrical signals may be radio frequency signals in the form of intermittent pulses.

According to the present disclosure, desirably, the radio frequency signals may be controlled by signal application time in the adjustment of the size of energy transferred.

According to the present disclosure, desirably, the radio frequency signals may be controlled to control radio frequency, application time, pause time, and a repetition rate, control desired frequency, application time, and pause time, and thus adjust the amount of energy applied.

According to the present disclosure, desirably, the negative pressure-forming portions may be grooves that are formed around the needles on the end of the handpiece assembly and connected to a suction part by means of a suction hose, the grooves having the shapes of long ditches or manholes positioned at given intervals, and the suction hose being closed by a suction valve if the suction of the suction part is kept.

According to the present disclosure, desirably, the handpiece assembly may further include metal members located on the end thereof in such a way as to come into close contact with the skin and cool the skin.

According to the present disclosure, desirably, the metal members may be arranged continuously in such a way as to surround the outsides of the negative pressure-forming portions, and otherwise, the metal members may be arranged in the form of a lattice on the end of the handpiece assembly.

According to the present disclosure, desirably, the metal members may be located on the end of the handpiece assembly to transfer cooling air to the skin using a Peltier device or refrigerant compression.

According to the present disclosure, desirably, the handpiece assembly may further include cooling gas emission holes formed on the end thereof to emit cooling gas to the skin around the needles.

According to the present disclosure, desirably, the controller may include a gas emission control device consisting of a valve, a storage tank, and a control unit that are adapted to control an amount of cooling gas emitted through the cooling gas emission holes and emission time of the cooling gas.

According to the present disclosure, desirably, the cooling gas emission time of the gas emission control device may be controlled by the controller, and given periods of cooling gas emission time may be controlled before the penetration of the needles, after the penetration of the needles, and during the penetration of the needles to thus minimize the skin pain caused by the penetration of the needles.

According to the present disclosure, desirably, given periods of cooling gas emission time of the gas emission control device may be controlled before the application of the electrical signals, after the application of the electrical signals, and during the application of the electrical signals by the controller to thus minimize the skin pain and damage caused by the application of the electrical signals.

### BRIEF DESCRIPTION OF THE DRAWINGS

The above and other objects, features and advantages of the present disclosure will be apparent from the following detailed description of the embodiments of the disclosure in conjunction with the accompanying drawings, in which:

FIG. 1 is a schematic block diagram showing a skin treatment device according to an embodiment of the present disclosure;

FIG. 2 is a perspective view showing a handpiece assembly of the skin treatment device according to the embodiment of the present disclosure;

FIG. 3 is a front view showing the handpiece assembly of the skin treatment device according to the embodiment of the present disclosure;

FIG. 4 is a side sectional view showing the handpiece assembly of the skin treatment device according to the embodiment of the present disclosure;

FIG. 5 is a plan sectional view showing the handpiece assembly of the skin treatment device according to the embodiment of the present disclosure;

FIG. 6 is an exploded perspective view showing the handpiece assembly of the skin treatment device according to the embodiment of the present disclosure;

FIG. 7 is an exploded perspective view showing a needle assembly of the skin treatment device according to the embodiment of the present disclosure;

FIG. 8 is a side sectional view showing a state where the needle assembly is coupled to a contact cap according to the embodiment of the present disclosure;

FIG. 9 is a front sectional view showing the state where the needle assembly is coupled to the contact cap according to the embodiment of the present disclosure;

FIG. 10 is an exploded perspective view showing a driver and a locker of the skin treatment device according to the embodiment of the present disclosure;

FIG. 11 is an exploded perspective view showing the driver of the skin treatment device according to the embodiment of the present disclosure;

FIG. 12 is an exploded perspective view showing the locker of the skin treatment device according to the embodiment of the present disclosure;

FIG. 13 is a schematic plan view showing a state where a negative pressure-forming portion is formed on the contact cap located on the end of the handpiece assembly of the skin treatment device according to the embodiment of the present disclosure;

FIG. 14 is a schematic side sectional view showing a state where the skin is sucked by a negative pressure according to the embodiment of the present disclosure;

FIG. 15 is a schematic side sectional view showing a state where the negative pressure is not applied by means of a closed suction valve according to the embodiment of the present disclosure;

FIG. 16 is a schematic plan view showing a state where metal members are located on the contact cap located on the end of the handpiece assembly of the skin treatment device according to the embodiment of the present disclosure;

FIG. 17 is a schematic plan view showing a state where the metal members are cooled by means of a Peltier device according to the embodiment of the present disclosure;

FIG. 18 is a schematic plan view showing a state where cooling gas emission holes are formed on the contact cap located on the end of a handpiece assembly of a skin treatment device according to another embodiment of the present disclosure; and

FIG. 19 is a graph showing cooling gas emission time through the cooling gas emission holes according to another embodiment of the present disclosure.

### DETAILED DESCRIPTION OF THE PREFERRED EMBODIMENT

Hereinafter, an embodiment of the present disclosure will be explained in detail with reference to the attached drawings.

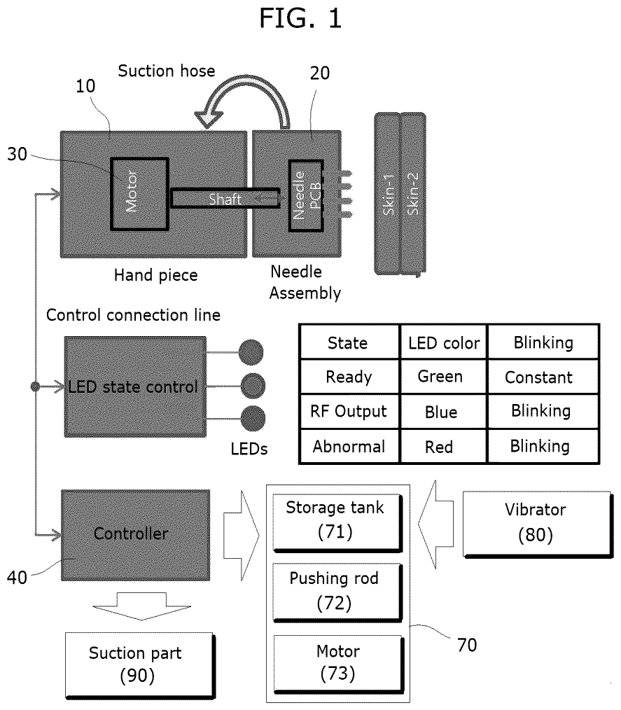

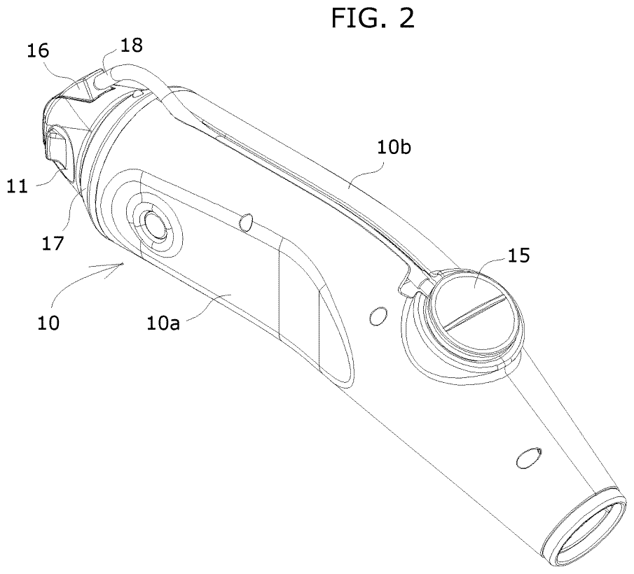

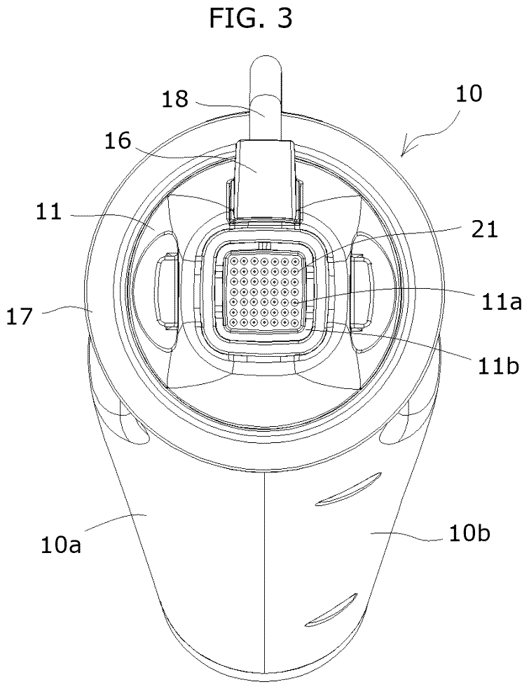

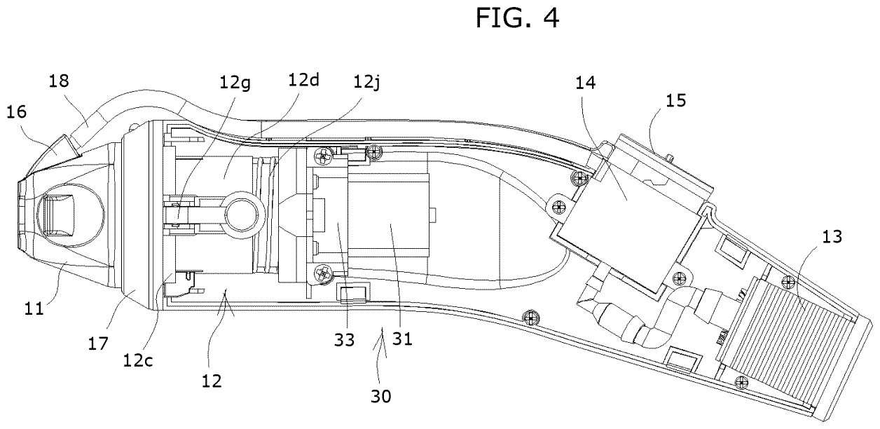

FIG. 1 is a schematic block diagram showing a skin treatment device according to an embodiment of the present disclosure, FIG. 2 is a perspective view showing a handpiece assembly of the skin treatment device according to the embodiment of the present disclosure, FIG. 3 is a front view showing the handpiece assembly of the skin treatment device according to the embodiment of the present disclosure, and FIG. 4 is a side sectional view showing the handpiece assembly of the skin treatment device according to the embodiment of the present disclosure.

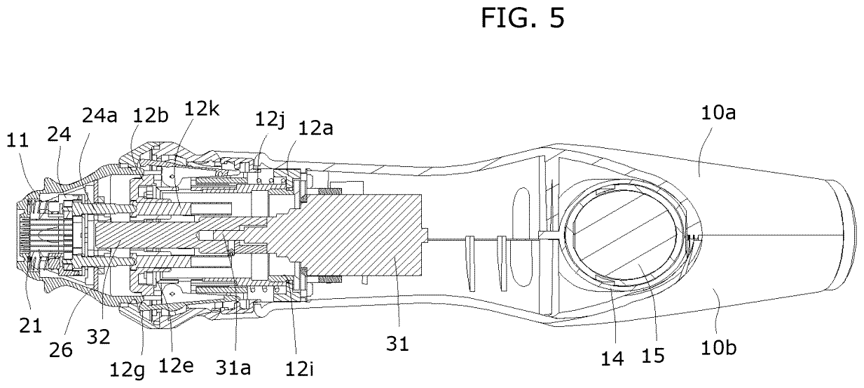

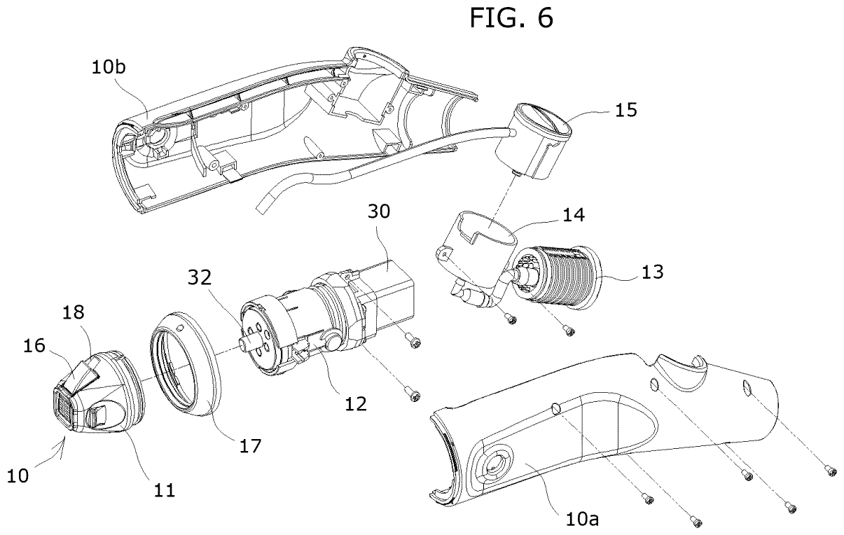

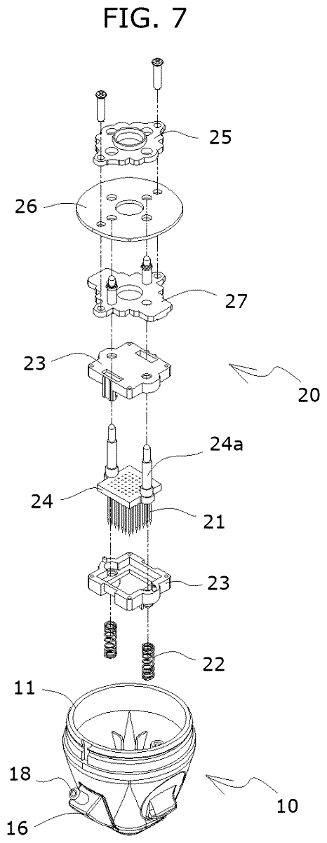

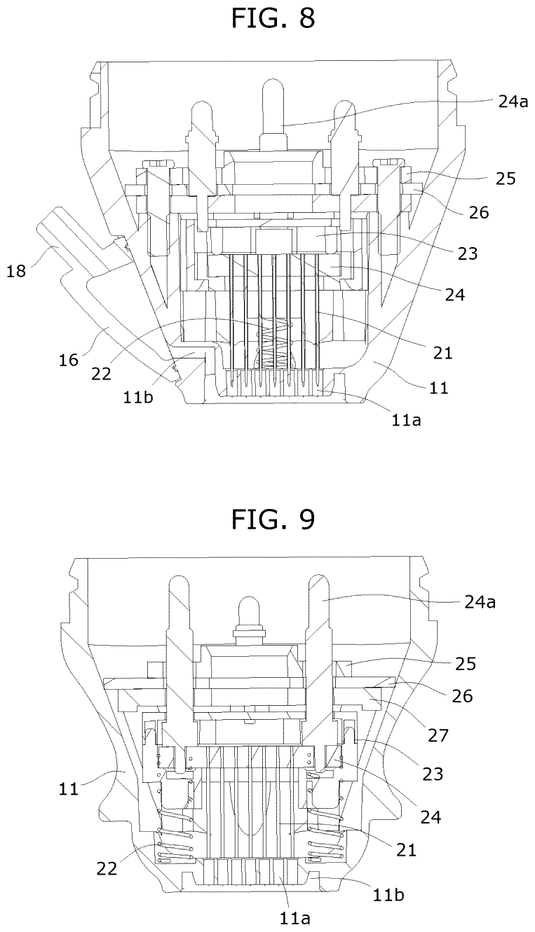

FIG. 5 is a plan sectional view showing the handpiece assembly of the skin treatment device according to the embodiment of the present disclosure, FIG. 6 is an exploded perspective view showing the handpiece assembly of the skin treatment device according to the embodiment of the present disclosure, FIG. 7 is an exploded perspective view showing a needle assembly of the skin treatment device according to the embodiment of the present disclosure, FIG. 8 is a side sectional view showing a state where the needle assembly is coupled to a contact cap according to the embodiment of the present disclosure, and FIG. 9 is a front sectional view showing the state where the needle assembly is coupled to the contact cap according to the embodiment of the present disclosure.

Referring to FIGS. 1 to 9, a skin treatment device according to an embodiment of the present disclosure largely includes a handpiece assembly 10, a needle assembly 20, a driver 30, a controller 40, negative pressure-forming portions 50, and metal members 60.

The handpiece assembly 10 is connected to the controller 40 through a control cable or the like and includes housings 10a and 10b and a contact cap 11 coupled to the front ends of the housings 10a and 10b and having needle through holes 11a and suction holes 11b formed thereon.

The housings 10a and 10b are coupled to each other in such a way as to have an internal space therebetween, and therefore, in the internal space formed by the housings 10a and 10b are received the driver 30 for controlling the movement of the needle assembly 20 received inside the contact cap 11 and a locker 12 for connecting the needle assembly 20 sucking the skin by a negative pressure to the driver 30, while locking or unlocking the driver 30 thereonto or therefrom.

If the needle assembly 20 is received in the contact cap 11 located on the end of the handpiece assembly 10, the needle assembly 20 is released from a restrainedly coupled state to the locker 12.

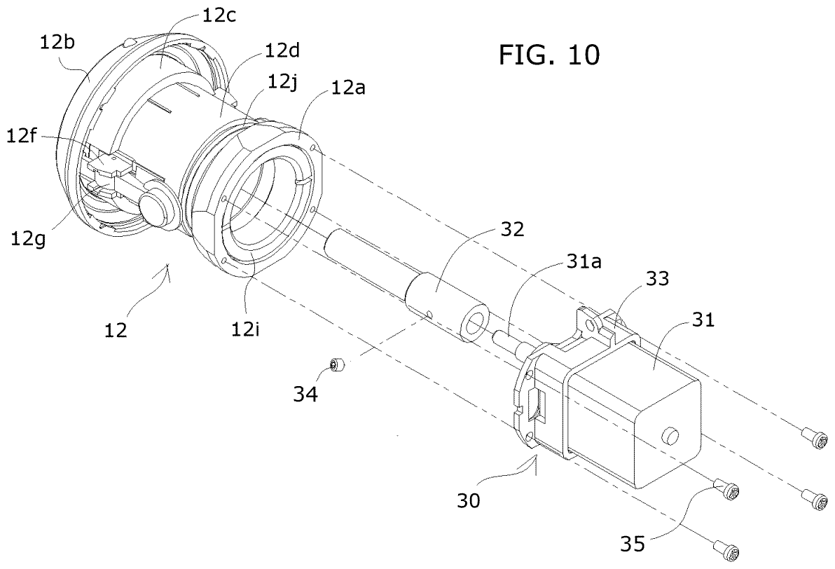

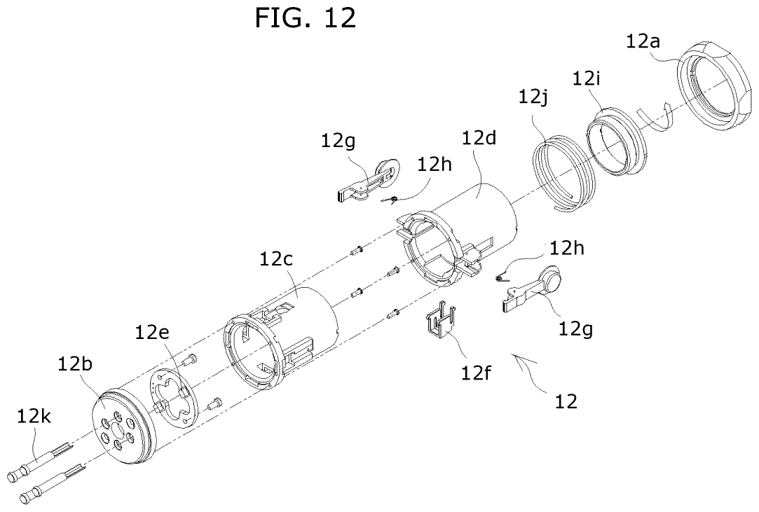

As shown in FIGS. 10 and 12, the locker 12 includes a base 12a, a locking cover 12b, and a barrel 12c, a slider 12d, and an LED 12e located between the base 12a and the locking cover 12b, and in this case, the slider 12 d has locking hooks 12g and torsion springs 12h coupled thereto by means of supports 12f. Further, a fixing nut 12i and a slide spring 12j are connected to the base 12a, and terminal pins 12k are connected to the locking cover 12b.

If the needle assembly 20 is fixed to the handpiece assembly 10, therefore, the end of the contact cap 11, to which the needle assembly 20 is coupled, pushes the barrel 12c and is then coupled thereto, and the barrel 12c pushes the end of the contact cap 11 in the opposite direction to the direction pushed thereof by means of the slide springs 12j located therebehind. If the end of the contact cap 11 pushes by a given depth or more, the locking hooks 12g protrude from the slider 12d by means of the torsion springs 12h, so that they are pushed and locked onto the projection of the end of the contact cap 11 and thus hold the contact cap 11.

Further, an output connector 13 is coupled to the rears of the housings 10a and 10b in such a way as to transmit a radio frequency to the needle assembly 20, and the output connector 13 is connected to an operating module so that the radio frequency is transmitted to the needle assembly 20. The output connector 13 is an electrical connector structure having 13 pins for radio frequency application, motor control, and detachable attachment of communication lines.

Furthermore, a filter case 14 and a filter unit 15 received in the filter case 14 are located inside the housings 10a and 10b. One end of the filter unit 15 is connected to, for example, a suction pump of the operating module, and the other end is connected to an induction unit 16.

In this case, the filter unit 15 serves to filter blood, body fluids, discharge, and tissue scraps sucked during a treatment, thereby preventing them from entering the driver 30.

The induction unit 16 is located on the outer peripheral surface of the contact cap 11 and connected to the negative pressure-forming portions 50, and the contact cap 11 is separably located on the front ends of the housings 10a and 10b by means of a connector 17, while locating the needle assembly 20 therein.

In this case, the connector 17 serves to guide the coupling of the needle assembly 20 to the contact cap 11, while coupling the housings 10a and 10b to each other.

The end of the contact cap 11 has the shape of a polygon or circle so that it can be brought into contact with an area around the skin to be treated.

The contact cap 11 may have various shapes according to the structure of the needle assembly 20, while having a receiving space formed therein and the induction unit 16 formed on the outer peripheral surface thereof.

The needle assembly 20 serves to suck the skin by means of the negative pressure and is separably coupled to the handpiece assembly 10. The needle assembly 20 includes a plurality of needles 21 exposedly passing through the needle through holes 11a of the contact cap 11 located on the end of the handpiece assembly 10 and a pair of bases 23 loaded with springs 22 in such a way as to move forward and backward.

If the needles 21 and the bases 23 of the needle assembly 20 move forward and backward, the springs 22 allow the needles 21 to exposedly pass through the needle through holes 11a formed on the end of the contact cap 11 so that the needles 21 are exposed in the direction parallel to the moving direction of the needle assembly 20.

In detail, the bases 23 and a needle substrate 24 as forward and backward moving bodies of the needle assembly 20 are pushed in the opposite direction to the contact cap 11 through the tension of the springs 22, and they are pushed toward the surface of the contact cap 11 through a push bar 32 as will be discussed later of the driver 30 so that the needles 21 exposedly pass through the needle through holes 11a of the surface of the contact cap 11. If the push bar 32 moves backward by means of the backward movement of the driver 30, the springs 22 apply forces for returning the bases 23 and the needle substrate 24 to their original position.

In this case, the needle substrate 24 electrically connects the radio frequency signals received through pogo pins 24a connected to the handpiece assembly 10 to the needles 21 so that the radio frequency signals are transmitted to the needles 21. Even though not shown, an LED may be mounted on the needle substrate 24.

In this case, the bases 23 are one pair of facing structures received in the contact cap 11, and the springs 22 for absorbing the impacts applied to the needle substrate 24 are coupled to the bases 23. Of course, the needles 21 are coupled to one surface of the needle substrate 24, and the bases 23 are restricted in movements by means of a movement restriction member 27 connected to the locker 12.

In this case, the movement restriction member 27 serves to allow the needle substrate 24 to have different resistance values according to the number of pins, if the number of needle pins of the needle substrate 24 is 14, 25, 36, or 49, so that electric currents flowing through the two pogo pins 24a are differently restricted. This is performed through the recognition of the number of pins.

Therefore, the bases 23 and the needle substrate 24 to which the needles 21 are coupled can move forward and backward according to the operation of the driver 30.

Further, the needle assembly 20 includes a fixing member 25 for fixing the contact cap 11 thereto and a silicone cover 26 for separating the directions of the needles 21 therein from a connected portion to the handpiece assembly 10.

The silicone cover 26 is located between the fixing member 25 and the movement restriction member 27 in such a way as to keep air tightness therebetween and thus prevent the negative pressure formed in a space between the contact cap 11 and the skin through an induction tap 18 of the induction unit 16 from leaking through the needle through holes 11a. As shown in FIG. 7, a circular hole formed on the central portion of the silicone cover 26 comes into contact with a pushing rod 72 for pushing the bases 23 so that the needle assembly 20 moves forward, and further, the silicone cover 26 is desirably made of an expandable thin rubber or silicone material so that the remaining portion of the silicone cover 26 is fixed, but while the pushing rod 72 is pushed, the negative pressure is kept.

The entire portion except the ends of some or all of the needles 21 is insulatedly formed, and further, some of the needles 21 are exposed longer than the remaining needles 21 and thus different in skin penetration depths.

Further, some of the needles 21 are hollow so that they penetrate the skin and thus inject medicine into the skin, and in this case, the medicine is fed from a storage tank 71 of a medicine injection part 70 that is detachably mounted on the handpiece assembly 10. The pushing rod 72 is located in the storage tank 71 and pushes and injects the medicine, and the pushing rod 72 is coupled to a motor 73 and operates through the controller 40 connected thereto so that the medicine is pushed and injected at consistent time and speed.

In this case, the handpiece assembly 10 is provided with a vibrator 80 that applies vibrations so that the injected medicine uniformly spreads, and the vibrations include vibrations in the same directions as the exposed directions of the needles 21 and vibrations in diagonal directions with respect to the exposed directions of the needles 21. Their frequency is adjustable according to the control of the controller 40.

The needle assembly 20 includes a storage unit so that it has unique identifiable information in the form of a barcode or QR code, and the information stored in the storage unit includes a manufacturing date, the number of pins, the number of uses, an expiration date, and version information.

In this case, the LED 12e is built in the needle assembly 20 so that lighting is controlled by the signal provided from the controller 40 electrically connected to the needle assembly 20 to allow the light emitted from the LED 12e to be applied to the area to be treated through the contact cap 11, and therefore, the application of the electrical signal to the human body through the needles 21 is displayed.

As shown in FIG. 1, the LED 12e is appropriately set to allow the operating states of the skin treatment device to be recognized according to the light colors of the LED 12e. If green light is constantly emitted, it means that an output is ready, and if blue light blinks, it means that a radio frequency output is produced. Further, if red light blinks, it means that the skin treatment device is abnormal. Such control enables the skin treatment device to be used in safe and accurate ways.

The driver 30 is located inside the handpiece assembly 10 and thus serves to allow the needles 21 to exposedly pass through the needle through holes 11a formed on the end of the contact cap 11 by desired distances according to a given output signal of the controller 40.

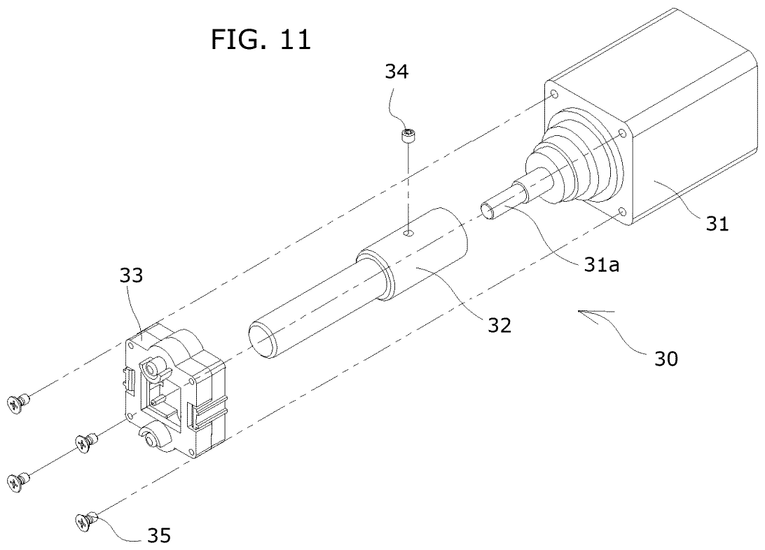

As shown in FIGS. 10 and 11, the driver 30 includes a step motor 31 having a driving shaft 31a, the push bar 32 coupled to the driving shaft 31a by means of a headless bolt 34 in such a way as to move the needle assembly 20 forward or backward, and a motor bracket 33 coupled to the front end of the step motor 31 by means of screws 35 in such a way as to pass the push bar 32 therethrough.

The controller 40 is electrically connected to the needles 21 located on the needle substrate 24.

In this case, the controller 40 adopts a bipolar method in which when the electrical signals are transmitted to the needles 21, positive poles are connected to some of the needles 21 and negative poles are connected to the remaining needles 21, and otherwise, the controller 40 adopts a monopolar method in which positive poles are connected to the plurality of needles 21 and negative poles are connected separately to a given area of the human body.

Therefore, the needles 21 exposedly pass through the needle through holes 11a by the given depths and thus apply the electrical signals through the controller 40, and in this case, the needles 21 have different depths on the same positions and thus reach one or more depths to apply the electrical signals. The needles 21 are electrically coupled to one another and thus apply the electrical signals from the controller 40 for a desired time, and therefore, the electrical signals produced from the controller 40 are radio frequency signals in the form of intermittent pulses.

That is, the radio frequency signals produced from the controller 40 are controlled by signal application time in the adjustment of the size of energy transferred, but radio frequency, application time, pause time, and a repetition rate are controlled to control desired frequency, application time, and pause time, thereby adjusting the amount of energy applied.

The negative pressure-forming portions 50 are formed around the needles 21 on the end of the contact cap 11 of the handpiece assembly 10 to suck the skin, while applying the negative pressure to a space made with the contact with the skin, so that while the needles 21 exposedly pass through the needle through holes 11a and thus penetrate the skin, the negative pressure-forming portions 50 serve to fix the skin thereto, thereby preventing the skin from being displaced.

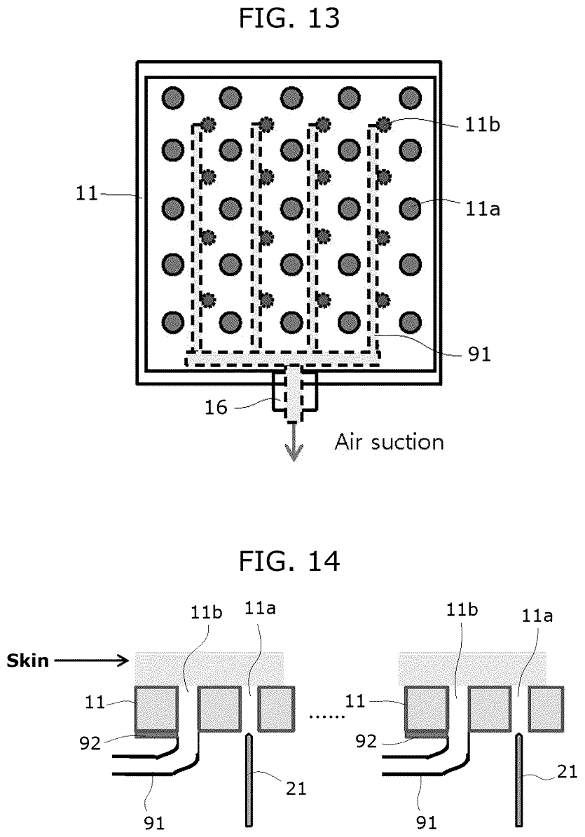

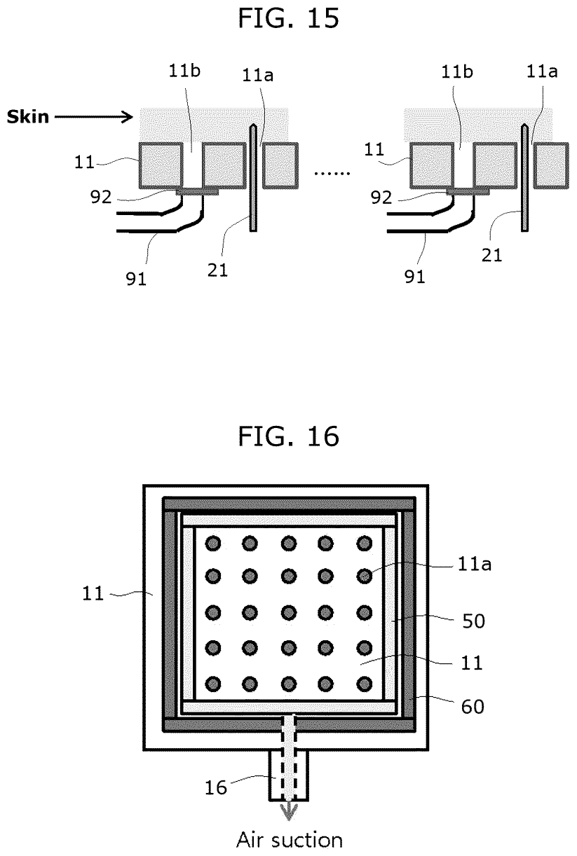

That is, as shown in FIGS. 13 to 15, the negative pressure-forming portions 50 are formed at the positions close to the needle through holes 11a of the contact cap 11 and connected to the induction unit 16 by means of a suction hose 91 so that the negative pressure-forming portions 50 apply the negative pressure to the space made with the contact with the skin by means of the suction force applied from the induction unit 16.

Therefore, the negative pressure-forming portions 50 are grooves that are formed on the contact cap 11 and connected to a suction part 90 by means of the suction hose 91 open by a suction valve 92, and in this case, the grooves may have the shapes of long ditches or manholes positioned at given intervals. Further, the suction hose 91 is closed by the suction valve 92 if the suction of the suction part 90 is kept.

The negative pressure space formed by the closed suction valve 92 allows the skin to be kept in a state of being brought into close contact with the contact cap 11, and even if the negative pressure on a given section is released, the negative pressure on other sections is kept, so that the suction is constantly performed to prevent the skin into which the needles 21 are inserted from moving, thereby ensuring safe treatment.

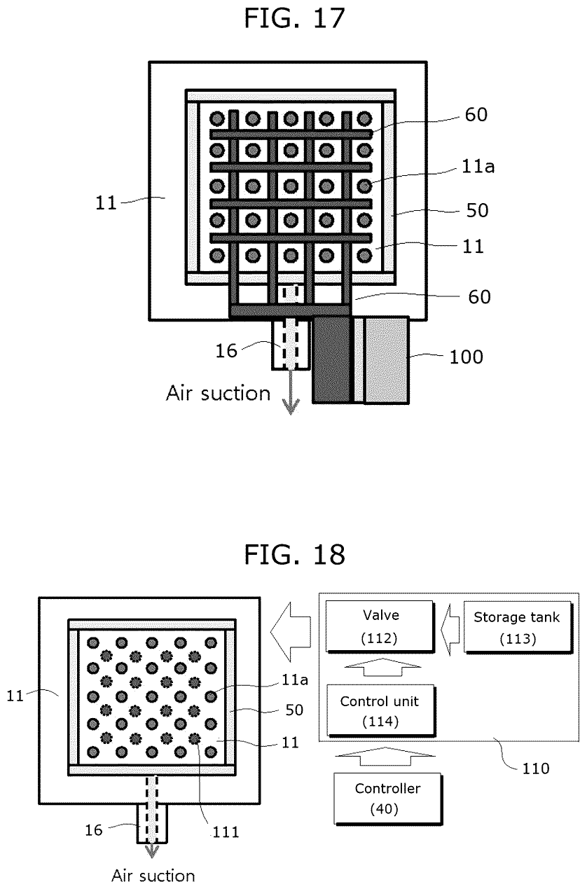

Further, as shown in FIGS. 16 and 17, the negative pressure-forming portions 50 have the shapes of edges surrounding the needle through holes 11a of the contact cap 11, and in this case, the metal members 60 are additionally applied. As shown in FIG. 16, the metal members 60 are arranged continuously in such a way as to surround the outsides of the negative pressure-forming portions 50, and otherwise, as shown in FIG. 17, the metal members 60 are arranged in the form of a lattice among the needle through holes 11a of the contact cap 11.

The metal members 60 are located on the end of the contact cap 11 and thus come into contact with the skin to allow the skin to be cool, and in this case, cooling air is transferred using a Peltier device 100 or refrigerant compression.

Like this, as shown in FIGS. 1 to 17, the skin treatment device according to the embodiment of the present disclosure applies the radio frequency to the large-area skin, thereby performing skin treatment through regeneration, provides the negative pressure-forming portions 50 on the end of the contact cap coming into contact with the skin, thereby preventing the skin into which the needles 21 are inserted from being displaced and controlling the insertion depths of the needles 21 consistently, and allows the metal members 60 located on the end of the contact cap 11 and coming into contact with the skin to cool the skin by means of the Peltier device 100, thereby preventing skin pain and damage from occurring upon the application of the electrical signals.

Further, if the insertion depths of the needles 21 are differently controlled or if the radio frequency application and the medicine injection are simultaneously performed, layers of the skin having different depths such as the papillary dermis and the reticular dermis can be simultaneously treated.

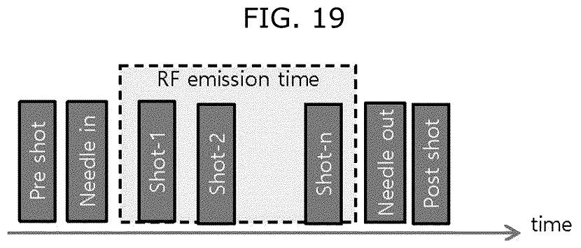

Further, FIGS. 18 and 19 show a skin treatment device according to another embodiment of the present disclosure. In this case, the skin treatment device according to another embodiment of the present disclosure includes cooling gas emission holes 111 formed on the end of the contact cap 11 to emit cooling gas to the skin around the needles 21 penetrating the skin, thereby preventing skin pain and damage from occurring upon the application of electrical signals through the controller 40.

That is, the controller 40 includes a gas emission control device 110 consisting of a valve 112, a storage tank 113, and a control unit 114 that are adapted to control an amount of cooling gas emitted through the cooling gas emission holes 111 and emission time of the cooling gas.

In this case, the cooling gas emission time of the gas emission control device 110 is controlled by the controller 40, and in this case, given periods of cooling gas emission time are controlled before the penetration of the needles 21, after the penetration of the needles 21, and during the penetration of the needles 21, thereby minimizing the skin pain caused by the penetration of the needles 21.

In this case, the cooling gas is compressed gas such as liquid carbon dioxide, Freon gas R134a2, nitrogen gas, and the like, and the storage tank 113 is connected to the cooling gas emission holes 111 through the valve 112.

Further, the cooling gas emission time of the gas emission control device 110 is controlled by the controller 40, and in this case, given periods of cooling gas emission time of the gas emission control device 110 are controlled before the application of the electrical signals, after the application of the electrical signals, and during the application of the electrical signals by the controller 40 to thus minimize the skin pain and damage caused by the application of the electrical signals, thereby enabling efficient temperature control.

In this case, the corresponding parts in the skin treatment device according to another embodiment of the present disclosure are indicated by corresponding reference numerals in the skin treatment device according to one embodiment of the present disclosure as shown in FIGS. 1 to 17 and the repeated explanation on the corresponding parts will be avoided.

As described above, the skin treatment device according to the present disclosure is configured to allow the handpiece assembly to have the negative pressure-forming portion formed on the end thereof in the form of the long ditch and/or manholes positioned at given intervals around the needles, thereby simultaneously treating the layers of the skin having different depths such as the papillary dermis and the reticular dermis and simultaneously performing the radio frequency application and the medicine injection, and configured to prevent the large-area skin from being displaced at the time when the needles penetrate the skin and allowing the penetration depths of the needles to be consistently controlled, thereby preventing skin pain and damage from occurring upon the radio frequency application and obtaining efficient skin treatment through the skin regeneration caused by the radio frequency application.

The foregoing description of the embodiments of the disclosure has been presented for the purpose of illustration; it is not intended to be exhaustive or to limit the disclosure to the precise forms disclosed.

Persons skilled in the relevant art can appreciate that many modifications and variations are possible in light of the above teachings. It is therefore intended that the scope of the disclosure be limited not by this detailed description, but rather by the claims appended hereto.

What is claimed is:

1. A skin treatment device comprising:
a handpiece assembly:
a needle assembly separably coupled to the handpiece assembly and having a plurality of needles exposedly passing through the end of the handpiece assembly and a pair of bases loaded with springs;
a driver located inside the handpiece assembly to allow the needles to exposedly pass through the end of the handpiece assembly by desired distances according to a given signal; and
a controller electrically connected to the needles of the needle assembly,
wherein the springs allow the needles to exposedly pass through the end of the handpiece assembly upon the movement of the needle assembly, thereby keeping conductive states of the needles, the needles being exposed in the parallel direction to the moving direction of the needle assembly,
the handpiece assembly comprises negative pressure-forming portions formed on the end thereof to suck the skin, while applying negative pressure to a space made with the contact with the skin around the needles, so that while the needles are exposed and thus penetrate the skin, the skin is fixed, without being displaced, and
the needle assembly further comprises a silicone cover adapted to keep air tightness and thus prevent the negative pressure formed through the negative pressure-forming portions from leaking to the spaces passing through the needles.

2. The skin treatment device according to claim 1, wherein some of the needles are exposed longer than the remaining needles and thus different in skin penetration depths.

3. The skin treatment device according to claim 1, wherein some of the needles are hollow so that the needles penetrate the skin and inject medicine into the skin.

4. The skin treatment device according to claim 3, wherein if some of the needles are hollow so that the needles inject the medicine into the skin, the medicine is fed from a storage tank of a medicine injection part that is detachably mounted on the handpiece assembly, pushed through a pushing rod located in the storage tank, and injected into the skin, the pushing rod being coupled to a motor of the medicine injection part in such a way as to operate through the controller connected thereto so that the medicine is pushed and injected at consistent time and speed.

5. The skin treatment device according to claim 4, wherein the handpiece assembly is provided with a vibrator that applies vibrations so that the injected medicine uniformly spreads.

6. The skin treatment device according to claim 5, wherein the vibrations include vibrations in the same directions as the exposed directions of the needles and vibrations in diagonal directions with respect to the exposed directions of the needles.

7. The skin treatment device according to claim 1, wherein the entire portion excepting the ends of some or all of the needles is insulatedly formed.

8. The skin treatment device according to claim 1, wherein in the application of electrical signals to the needles from the controller, the controller adopts a bipolar method in which positive poles are connected to some of the needles and negative poles are connected to the remaining needles.

9. The skin treatment device according to claim 1, wherein in the application of electrical signals to the needles from the controller, the controller adopts a monopolar method in which positive poles are connected to the plurality of needles and negative poles are connected separately to a given area of the human body.

10. The skin treatment device according to claim 1, wherein the handpiece assembly further comprises a locker adapted to restrict the needle assembly to allow the needle assembly to be coupled thereto and released from the coupled state.

11. The skin treatment device according to claim 1, wherein the needle assembly comprises a storage unit through which unique identifiable information is obtained, and the information stored in the storage unit comprises a manufacturing date, the number of pins, the number of uses, an expiration date, and version information.

12. The skin treatment device according to claim 11, wherein the needle assembly has the unique identifiable information in the form of a barcode or QR code.

13. The skin treatment device according to claim 1, wherein the handpiece assembly or the needle assembly has an LED built therein so that lighting is controlled by the signal provided from the controller electrically connected to the handpiece assembly or the needle assembly to allow the light emitted from the LED to be applied to the area to be treated through the end of the handpiece assembly, and the colors of the LED represent the operating states of the skin treatment device so that the application of the electrical signals to the human body through the needles is displayed.

14. The skin treatment device according to claim 1, wherein the needles are exposed by the given distances and thus apply the electrical signals to the skin, and the needles have different penetration depths on the same positions and thus reach one or more depths to apply the electrical signals.

15. The skin treatment device according to claim 1, wherein the needles are electrically coupled to one another and thus apply the electrical signals from the controller for a desired time to the skin, and the electrical signals are radio frequency signals in the form of intermittent pulses.

16. The skin treatment device according to claim 15, wherein the radio frequency signals are controlled by signal application time in the adjustment of the size of energy transferred.

17. The skin treatment device according to claim 15, wherein the radio frequency signals are controlled to control radio frequency, application time, pause time, and a repetition rate, control desired frequency, application time, and pause time, and adjust the amount of energy applied.

18. The skin treatment device according to claim 1, wherein the negative pressure-forming portion is a groove that is formed around the needles on the end of the handpiece assembly and connected to a suction part by means of a suction hose, the groove having the shape of a long ditch or manholes positioned at given intervals, and the suction hose being closed by a suction valve if the suction of the suction part is kept.

19. The skin treatment device according to claim 1, wherein the handpiece assembly further comprises metal members located on the end thereof in such a way as to come into close contact with the skin and cool the skin.

20. The skin treatment device according to claim 19, wherein the metal members are arranged continuously in such a way as to surround the outside of the negative pressure-forming portion.

21. The skin treatment device according to claim 19, wherein the metal members are arranged in the form of a lattice among needle through holes formed on a contact cap located on the end of the handpiece assembly.

22. The skin treatment device according to claim 19, wherein the metal members are located on the end of the handpiece assembly to transfer cooling air to the skin using a Peltier device or refrigerant compression.

23. A skin treatment device comprising:
a handpiece assembly:
a needle assembly separably coupled to the handpiece assembly and having a plurality of needles exposedly passing through the end of the handpiece assembly and a pair of bases loaded with springs;
a driver located inside the handpiece assembly to allow the needles to exposedly pass through the end of the handpiece assembly by desired distances according to a given signal; and
a controller electrically connected to the needles of the needle assembly,
wherein the springs allow the needles to exposedly pass through the end of the handpiece assembly upon the movement of the needle assembly, thereby keeping conductive states of the needles, the needles being exposed in the parallel direction to the moving direction of the needle assembly, and
the handpiece assembly comprises: negative pressure-forming portions formed on the end thereof to suck the skin, while applying negative pressure to a space made with the contact with the skin around the needles so that while the needles are exposed and thus penetrate the skin, the skin is fixed, without being displaced; and
cooling gas emission holes formed on the end thereof to emit cooling gas to the skin around the needles.

24. The skin treatment device according to claim 23, wherein the controller comprises a gas emission control device consisting of a valve, a storage tank, and a control unit that are adapted to control an amount of cooling gas emitted through the cooling gas emission holes and emission time of the cooling gas.

25. The skin treatment device according to claim 24, wherein the cooling gas emission time of the gas emission control device is controlled by the controller, and given periods of cooling gas emission time are controlled before the penetration of the needles, after the penetration of the needles, and during the penetration of the needles to thus minimize the skin pain caused by the penetration of the needles.

26. The skin treatment device according to claim 24, wherein given periods of cooling gas emission time of the gas emission control device are controlled by the controller before the application of the electrical signals, after the application of the electrical signals, and during the application of the electrical signals to thus minimize the skin pain and damage caused by the application of the electrical signals.
